SEQUENCE LISTING (6 sequences)

SEQ ID NO: 1 - protein, 3423 aa, Unknown
  Zika virus
MKNPKKKSGGFRIVNMLKRGVARVSPFGGLKRLPAGLLLGHGPIRMVLAILAFLRFTAIKPSLGLINRWGSVGKKEAMEIIKKFKKDLAAMLRIINARKEKKRRGADTSVGIVGLLLTTAMAAEVTRRGSAYYMYLDRNDAGEAISFPTTLGMNKCYIQIMDLGHTCDATMSYECPMLDEGVEPDDVDCWCNTTSTWVVYGTCHHKKGEARRSRRAVTLPSHSTRKLQTRSQTWLESREYTKHLIRVENWIFRNPGFALAAAAIAWLLGSSTSQKVIYLVMILLIAPAYSIRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKSLFGGMSWFSQILIGTLLMWLGLNAKNGSISLMCLALGGVLIFLSTAVSADVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSTDHMDHFSLGVLVILLMVQEGLKKRMTTKIIISTSMAVLVAMILGGFSMSDLAKLAILMGATFAEMNTGGDVAHLALIAAFKVRPALLVSFIFRANWTPRESMLLALASCLLQTAISALEGDLMVLINGFALAWLAIRAMVVPRTDNITLAILAALTPLARGTLLVAWRAGLATCGGFMLLSLKGKGSVKKNLPFVMALGLTAVRLVDPINVVGLLLLTRSGKRSWPPSEVLTAVGLICALAGGFAKADIEMAGPMAAVGLLIVSYVVSGKSVDMYIERAGDITWEKDAEVTGNSPRLDVALDESGDFSLVEDDGPPMREIILKVVLMTICGMNPIAIPFAAGAWYVYVKTGKRSGALWDVPAPKEVKKGETTDGVYRVMTRRLLGSTQVGVGVMQEGVFHTMWHVTKGSALRSGEGRLDPYWGDVKQDLVSYCGPWKLDAAWDGHSEVQLLAVPPGERARNIQTLPGIFKTKDGDIGAVALDYPAGTSGSPILDKCGRVIGLYGNGVVIKNGSYVSAITQGRREEETPVECFEPSMLKKKQLTVLDLHPGAGKTRRVLPEIVREAIKTRLRTVILAPTRVVAAEMEEALRGLPVRYMTTAVNVTHSGTEIVDLMCHATFTSRLLQPIRVPNYNLYIMDEAHFTDPSSIAARGYISTRVEMGEAAAIFMTATPPGTRDAFPDSNSPIMDTEVEVPERAWSSGFDWVTDHSGKTVWFVPSVRNGNEIAACLTKAGKRVIQLSRKTFETEFQKTKHQEWDFVVTTDISEMGANFKADRVIDSRRCLKPVILDGERVILAGPMPVTHASAAQRRGRIGRNPNKPGDEYLYGGGCAETDEDHAHWLEARMLLDNIYLQDGLIASLYRPEADKVAAIEGEFKLRTEQRKTFVELMKRGDLPVWLAYQVASAGITYTDRRWCFDGTTNNTIMEDSVPAEVWTRHGEKRVLKPRWMDARVCSDHAALKSFKEFAAGKRGAAFGVMEALGTLPGHMTERFQEAIDNLAVLMRAETGSRPYKAAAAQLPETLETIMLLGLLGTVSLGIFFVLMRNKGIGKMGFGMVTLGASAWLMWLSEIEPARIACVLIVVFLLLVVLIPEPEKQRSPQDNQMAIIIMVAVGLLGLITANELGWLERTKSDLSHLMGRREEGATIGFSMDIDLRPASAWAIYAALTTFITPAVQHAVTTSYNNYSLMAMATQAGVLFGMGKGMPFYAWDFGVPLLMIGCYSQLTPLTLIVAIILLVAHYMYLIPGLQAAAARAAQKRTAAGIMKNPVVDGIVVTDIDTMTIDPQVEKKMGQVLLIAVAVSSAILSRTAWGWGEAGALITAATSTLWEGSPNKYWNSSTATSLCNIFRGSYLAGASLIYTVTRNAGLVKRRGGGTGETLGEKWKARLNQMSALEFYSYKKSGITEVCREEARRALKDGVATGGHAVSRGSAKLRWLVERGYLQPYGKVIDLGCGRGGWSYYAATIRKVQEVKGYTKGGPGHEEPVLVQSYGWNIVRLKSGVDVFHMAAEPCDTLLCDIGESSSSPEVEEARTLRVLSMVGDWLEKRPGAFCIKVLCPYTSTMMETLERLQRRYGGGLVRVPLSRNSTHEMYWVSGAKSNTIKSVSTTSQLLLGRMDGPRRPVKYEEDVNLGSGTRAVVSCAEAPNMKIIGNRIERIRSEHAETWFFDENHPYRTWAYHGSYEAPTQGSASSLINGVVRLLSKPWDVVTGVTGIAMTDTTPYGQQRVFKEKVDTRVPDPQEGTRQVMSMVSSWLWKELGKHKRPRVCTKEEFINKVRSNAALGAIFEEEKEWKTAVEAVNDPRFWALVDKEREHHLRGECQSCVYNMMGKREKKQGEFGKAKGSRAIWYMWLGARFLEFEALGFLNEDHWMGRENSGGGVEGLGLQRLGYVLEEMSRIPGGRMYADDTAGWDTRISRFDLENEALITNQMEKGHRALALAIIKYTYQNKVVKVLRPAEKGKTVMDIISRQDQRGSGQVVTYALNTFTNLVVQLIRNMEAEEVLEMQDLWLLRRSEKVTNWLQSNGWDRLKRMAVSGDDCVVKPIDDRFAHALRFLNDMGKVRKDTQEWKPSTGWDNWEEVPFCSHHFNKLHLKDGRSIVVPCRHQDELIGRARVSPGAGWSIRETACLAKSYAQMWQLLYFHRRDLRLMANAICSSVPVDWVPTGRTTWSIHGKGEWMTTEDMLVVWNRVWIEENDHMEDKTPVTKWTDIPYLGKREDLWCGSLIGHRPRTTWAENIKNTVNMVRRIIGDEEKYMDYLSTQVRYLGEEGSTPGVL

SEQ ID NO: 2 - DNA, 1428 bp, Artificial Sequence
  DNA Sequence of ExpreS2ion Ev1
atgaagctgtgcatcctgctggccgtggtggccttcgtgggactgagtctgggacgctgcatcggcgtgtccaaccgcgatttcgtggagggcatgagcggcggaacctgggtggacgtggtgctggagcatggcggatgcgtgaccgtgatggcccaggataagcccaccgtggatatcgagctcgtgaccaccaccgtgtcgaacatggccgaagtgcgcagctactgctacgaggccagcatcagcgatatggccagcgatagccgctgcccaacccagggcgaggcctacctggataagcagagcgatacccagtacgtgtgcaagcgcaccctggtggatcgcggctggggaaatggatgcggcctgttcggaaagggcagcctcgtgacctgcgccaagttcgcctgcagcaagaagatgaccggcaagagcatccagcccgagaacctggagtaccgcatcatgctgagcgtgcacggctcccagcacagcggcatgatcgtgaacgataccggccacgagaccgatgagaaccgcgccaaggtggagatcacccccaatagtccacgcgccgaggccacgctgggaggatttggaagtctgggcctggattgcgagccacgcaccggactggatttcagcgatctgtactacctgaccatgaacaacaagcactggctggtgcacaaggagtggttccacgatatccccctgccctggcacgccggagccgataccggaaccccacactggaacaacaaggaggccctggtggagttcaaggatgcccacgccaagcgccagaccgtggtggtgctgggaagccaggagggcgccgtgcataccgccctggccggagccctggaggccgagatggatggcgccaagggacgcctgagtagcggccatctgaagtgccgcctgaagatggataagctgcgcctgaagggcgtgtcctacagcctgtgcaccgccgccttcaccttcaccaagatcccagccgagaccctgcacggcaccgtgacggtggaggtgcagtatgccggaaccgatggcccctgcaaggtgccagcccagatggccgtggacatgcagaccctgaccccagtgggccgcctgatcaccgccaatccagtgatcaccgagagcaccgagaacagcaagatgatgctggagctggatccccccttcggcgattcctacatcgtgatcggcgtgggcgagaagaagatcacccaccactggcaccgcagcggcagcaccattggaaaggccttcgaggccaccgtgcgcggagccaagcgcatggccgtgctgggcgataccgcctgggatttcggaagcgtgggaggcgccctgaacagcctgggcaagggcattcaccagatcttcggagccgcctttaaggagcccgaggcctaa

SEQ ID NO: 3 - protein, 475 aa, Artificial Sequence
  Amino Acid Sequence of ExpreS2ion Ev1
MKLCILLAVVAFVGLSLGRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSGSTIGKAFEATVRGAKRMAVLGDTAWDFGSVGGALNSLGKGIHQIFGAAFKEPEA

SEQ ID NO: 4 - protein, 353 aa, Artificial Sequence
  Zika NS1 sequence
VGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGS

SEQ ID NO: 5 - protein, 534 aa, Artificial Sequence
  Expressed in the context of the Zika NS1 construct
MKLCILLAVVAFVGLSLGWSHPQFEKGGGSGGGSGGSSAWSHPQFEKSRSRGSLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGRDDLPSLPRENLYFQGVGCSVDFSKKETRCGTGVFVYNDVEAWRDRYKYHPDSPRRLAAAVKQAWEDGICGISSVSRMENIMWRSVEGELNAILEENGVQLTVVVGSVKNPMWRGPQRLPVPVNELPHGWKAWGKSYFVRAAKTNNSFVVDGDTLKECPLKHRAWNSFLVEDHGFGVFHTSVWLKVREDYSLECDPAVIGTAVKGKEAVHSDLGYWIESEKNDTWRLKRAHLIEMKTCEWPKSHTLWTDGIEESDLIIPKSLAGPLSHHNTREGYRTQMKGPWHSEELEIRFEECPGTKVHVEETCGTRGPSLRSTTASGRVIEEWCCRECTMPPLSFRAKDGCWYGMEIRPRKEPESNLVRSMVTAGSGAHIVMVDAYKPTK

SEQ ID NO: 6 - protein, 424 aa, Artificial Sequence
  Amino Acid Sequence of ExpreS2ion Ev2
MKLCILLAVVAFVGLSLGRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFACSKKMTGKSIQPENLEYRIMLSVHGSQHSGMIVNDTGHETDENRAKVEITPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLSSGHLKCRLKMDKLRLKGVSYSLCTAAFTFTKIPAETLHGTVTVEVQYAGTDGPCKVPAQMAVDMQTLTPVGRLITANPVITESTENSKMMLELDPPFGDSYIVIGVGEKKITHHWHRSEPEA

(19) United States
(12) Patent Application Publication
(10) Pub. No.: US 2019/0358313 A1
(43) Pub. Date: Nov. 28, 2019

(54) NEW FLAVIVIRUS VACCINE

(71) Applicant: EXPRES2ION BIOTECHNOLOGIES APS, Hørsholm (DK)

(72) Inventors: Willem Adriaan De Jongh, Holte (DK); Teit Max Moscote Søgaard, Kokkedal (DK); Thomas Dan Jørgensen, Helsingor (DK)

(21) Appl. No.: 16/472,414

(22) PCT Filed: Dec. 22, 2017
(86) PCT No.: PCT/EP2017/084527

§ 371 Date: Jun. 21, 2019

(30) Foreign Application Priority Data
Dec. 23, 2016 (EP) 16206717.7

(51) Int. Cl. A61K 39/12; C07K 16/10; C07K 14/18

(52) U.S. Cl. CPC A61K 39/12; C07K 14/1825; C07K 16/1081

(57) ABSTRACT

The present invention relates to polypeptides suitable for protection against and diagnosis of the conditions caused by flavivirus infections. More specifically, the invention concerns subunits of the zika virus envelope glycoprotein E secreted as mature recombinantly produced proteins from eucaryotic cells, such as from insect cells. Additional viral proteins or subunits, also produced in this way, provide additional active ingredients. These protein subunits, alone or in combination including combination with additional viral-derived peptides are protective against infection by flavivirus, such as zika virus, raise antibodies useful in immunization, and are useful in diagnosis of infection by the virus.

### FIELD OF THE INVENTION

The present invention relates to polypeptides suitable for protection against and diagnosis of the conditions caused by flavivirus infections. More specifically, the invention concerns subunits of the zika virus envelope glycoprotein E secreted as mature recombinantly produced proteins from eucaryotic cells, such as from insect cells. Additional viral proteins or subunits, also produced in this way, provide additional active ingredients. These protein subunits, alone or in combination including combination with additional viral-derived peptides are protective against infection by flavivirus, such as zika virus, raise antibodies useful in immunization, and are useful in diagnosis of infection by the virus.

### BACKGROUND OF THE INVENTION

Until recently, Zika virus (ZIKV) was believed to cause only mild disease, but a growing body of evidence that Zika virus infection results neurologic complications —Guillain-Barré syndrome in infected patients and microcephaly in unborn babies—combined with the very rapid spread of the Zika virus has spurred a host of projects to make a Zika vaccine candidate. The majority of planned clinical trials are based on DNA vaccines or attenuated strains of Zika and only one clinical phase 1 trial is currently (August 2016) underway (GloPID-R 2016; Maharajan et al. 2016).

In July 2016 a clinical phase 1 trial was initiated to test clinical safety and immunogenicity of a Zika Virus DNA Vaccine. The primary outcome of this trial is expected in December 2017. (https://clinicaltrials.gov identifier: NCT02840487). No subunit based vaccines trials are currently ongoing.

There is a need in the art for efficient and improved vaccines against flavivirus infection, such as zika virus infections.

### OBJECT OF THE INVENTION

It is an object of embodiments of the invention to provide polypeptides suitable for protection against and diagnosis of the conditions caused by flavivirus infections, such as by zika virus.

It is a further object of embodiments of the invention to provide vaccine compositions suitable for protection against and treatment of conditions caused by flavivirus infections, such as by zika virus in humans.

### SUMMARY OF THE INVENTION

It has been found by the present inventor(s) that some parts of flaviviral envelope E protein may form multimers beyond dimers. It is envisioned that the E protein multimers are more immunogenic than E protein subunits only present as dimers.

Accordingly the present invention provides a unique human vaccine to protect against disease associated with flavivirus, such as zika virus infection. The vaccine is formed by a recombinant subunit protein derived from virus proteins, such as zika proteins. Suitably adjuvants may be combined with the antigenic polypeptides of the invention, such as an aluminum hydroxide adjuvant.

So, in a first aspect the present invention relates to an isolated polypeptide comprising flaviviral envelope E protein, such as Zika virus envelope E protein, which polypeptide form multimers.

In a second aspect the present invention relates to an isolated polypeptide comprising a flavivirus NS1 sequence, such as Zika NS1 sequence.

In a third aspect the present invention relates to an isolated polypeptide comprising a first part being a polypeptide comprising flaviviral envelope E protein, such as Zika virus envelope E protein, fused, such as genetically fused, to a second part being a polypeptide comprising a flavivirus NS1 sequence, such as Zika NS1 sequence.

It is to be understood that fusion proteins comprising flaviviral envelope E protein, such as Zika virus envelope E protein fused to a flavivirus NS1 sequence, such as Zika NS1 sequence need not contain multimeric forms of flaviviral envelope E protein, such as Zika virus envelope E protein.

In a further aspect the present invention relates to a composition comprising a polypeptide comprising flaviviral envelope E protein, such as Zika virus envelope E protein, which polypeptide form multimers in combination with a polypeptide comprising a flavivirus NS1 sequence, such as Zika NS1 sequence.

In a further aspect the present invention relates to a composition comprising a vaccine formulation comprising a flavivirus vaccine, such as a Zika vaccine, consisting of a Virus-like particles (VLP) or an attenuated or inactivated vaccine, or DNA vaccine, in combination with a polypeptide comprising a flavivirus NS1 sequence, such as Zika NS1 sequence.

In a further aspect the present invention relates to the isolated polypeptide according the invention, or a composition according to the invention for use in a vaccine.

In a further aspect the present invention relates to the isolated polypeptide according the invention, or a composition according to the invention for use diagnostic.

In a further aspect the present invention relates to a vaccine for the protection of a subject against flavivirus infection, such as zika virus infection, which vaccine contains, as an active ingredient, a polypeptide or a composition according to the invention. In some embodiments the vaccine further contains an adjuvant.

In a further aspect the present invention relates to a conjugate comprising 1) a polypeptide selected from a) a polypeptide comprising flaviviral envelope E protein, such as Zika virus envelope E protein or b) a polypeptide comprising a flavivirus NS1 sequence, such as Zika NS1 sequence; and 2) a Virus-like Particle, such as conjugated by an isopeptide bond, such as by a split-protein binding system, such as a Spycatcher-Spy tag binding system, or a SdyCatcher binding system. In some embodiments according to the present inventions, the polypeptides used in this conjugate is a polypeptide according to the invention, such as a polypeptide comprising flaviviral envelope E protein, such as Zika virus envelope E protein or a polypeptide comprising a flavivirus NS1 sequence, such as Zika NS1 sequence.

In a further aspect the present invention relates to a method for the protection of a subject against flavivirus infection, such as zika virus infection, which method comprises administering to a subject in need of such protection an effective amount of the vaccine of the invention.

In a further aspect the present invention relates to an expression system for the production of an isolated polypeptide according to the invention, which expression system comprises a first nucleotide sequence encoding said polypeptide and optionally comprising a second encoding nucleotide sequence positioned so as to produce a fusion protein wherein a secretory leader sequence is operably linked to the polypeptide, and optionally further comprising a third nucleotide sequence encoding a tag sequence for analysis and/or purification; said encoding sequences operably linked to control sequences capable of effecting expression of said encoding nucleotide sequences.

In a further aspect the present invention relates to eucaryotic host cell modified to contain the expression system of the invention.

### LEGENDS TO THE FIGURE

FIG. 1: Coomassie stained 10% SDS-PAGE. L: Loaded material, F.T.: unbound fraction, M: Marker. Lanes 1-7 are reduced samples. Lane 9 and 10 are unreduced samples from the pooled peaks.

FIG. 2: A 4-12% reduced SDS-PAGE gel was blotted to a nitrocellulose membrane and probed using a anti-Zika antibody. Shown is a merged image combining the prestained markers on the blotted membrane and the Immunoblot of samples identical to the ones shown in FIG. 1 lanes 1-7. L: loaded material, F.T. unbound fraction, M: marker.

FIG. 3: Vector for preparation of Sumo-ZikaNs1, pExpreS2-1.

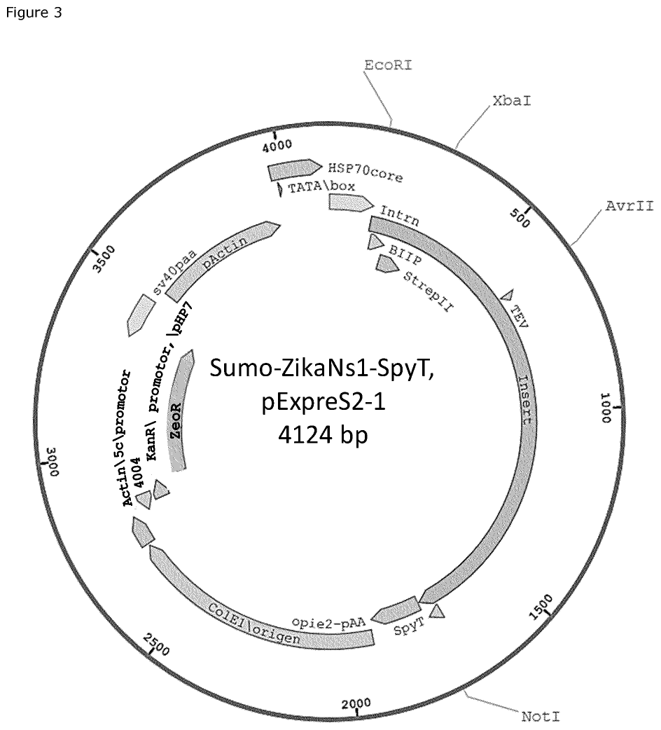

FIG. 4: Vector for preparation of Zika-Ev2, pExpreS2-PAC.

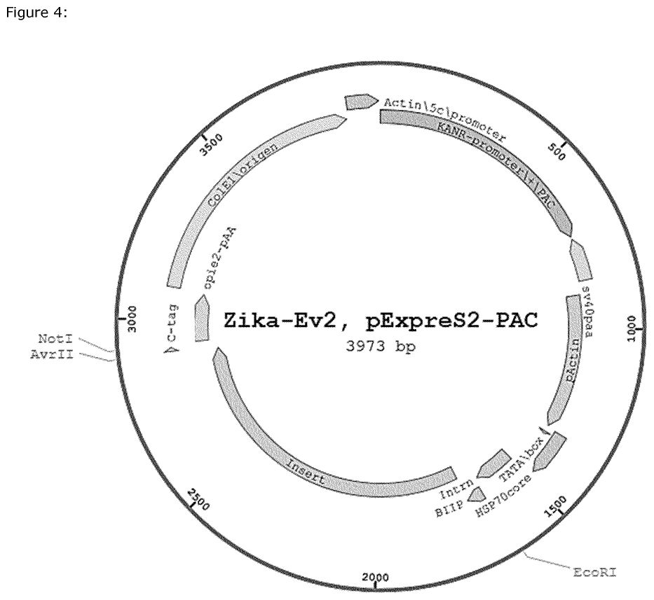

FIG. 5: PANEL A AND B ARE COOMASIE STAINED PAGE ANALYSIS. PANEL A SHOWS A BSA SUITABILITY MARKER (0.1 UG) IN LANE 1 AND THE PURIFIED ZIKA E-SPYCATCHER PROTEIN IN LANE 3. MARKER IN LANE 2. PANEL B SHOWS THE INPUT TO THE ISOPEPTIDE REACTION (LANE 2 AND 3), THE RESULT OF THE CONJUGATION (LANE 4) AND THE CENTRIFUGED ZIKA E PROTEIN-AP205 CONJUGATE [PRECIPITATION WOULD HAVE BEEN REVEALED BY DEPLETION OF THE BANDS ON THE GEL AFTER CENTRIFUGATION] (LANE 5) DETAILED

### DISCLOSURE OF THE INVENTION

The flavivirus, such as Zika virus vaccine of the present invention utilizes the flaviviral envelope E protein, such as Zika E recombinant subunit protein or alternatively flavivirus non-structural Protein NS1 subunit protein that may be produced by means of a cell culture expression system, such as based on Drosophila expression system, such as Drosophila Schneider 2 (S2) cells, such as the ExpreS2 Drosophila S2 cell line. The use of this system preferably results in recombinant subunit proteins that maintain native-like structure. The flavivirus E, such as Zika E recombinant subunit proteins are designed to contain most part of the flavivirus glycoprotein, central and dimerization domains as well as the flavivirus envelope glycoprotein E, optionally also containing part of the glycoprotein E stem/anchor region, such as approximately half of the flavivirus envelope glycoprotein E stem/anchor region. The design allow for secretion of the recombinant protein into the extracellular medium, thus facilitating recovery. “Secretion” as used herein means the ability to be secreted, and typically secreted, from the transformed cells of the expression system.

Alternatively, the present invention utilizes the flavivirus non-structural Protein NS1 subunit proteins which construct may contain this entire subunit. The flavivirus non-structural Protein NS1 subunit protein may be used alone or in combination with the flaviviral envelope E protein, such as Zika E recombinant subunit proteins of the invention. In some embodiments the NS1 subunit protein used according to the present invention is a secreted protein, when expressed as described herein.

The vaccine formulation of the present invention may include an adjuvant that is suitable for human use. One suitable adjuvant is an aluminum-based adjuvant, such as aluminum hydroxide, aluminum phosphate, or a mixture thereof. (e.g., Alhydrogel™).

The present invention provides a means for preventing or attenuating disease that result from infection by flavivirus, such as zika virus. As used herein, a vaccine is said to prevent or attenuate a disease if administration of the vaccine to an individual results either in the total or partial immunity of the individual to the disease, or in the total or partial attenuation (i.e., suppression) of symptoms or conditions associated with the disease.

A composition or components of a vaccine is said to be “pharmaceutically acceptable” if its administration can be tolerated by a recipient patient. Such an agent is said to be administered in a “therapeutically effective amount” if the amount administered is physiologically significant to have measurable effect. An agent is physiologically significant if its presence results in a detectable change in the physiology of a recipient patient. In the present invention the detectable change in the recipient patient is the induction of antibodies against flavivirus, such as zika virus.

The vaccine of the invention can be used alone or in combination with other active vaccines such as those containing other active subunits to the extent that they become available. Corresponding or different subunits from one or several viruses or serotypes may be included in a particular formulation. The active vaccine of the invention may further comprise a pharmaceutically acceptable excipient. The therapeutic compositions of the described invention can be administered parenterally by subcutaneous, intramuscular, or intradermal injection; however, other systemic modes of administration may also be employed.

A multiple administration regimen may be utilized. A suitable administration regimen may use the compositions of the invention more than once to increase the levels and diversities of expression of the immunoglobulin repertoire expressed by the immunized subject. Typically, if multiple immunizations are given, they will be given one to two months apart, such as at 0, 1, and 2 months. Alternative immunization schedules may be at 0, 1 and 3 months, or 0, 1 and 6 months, or 0, 1, 12 months. Additional booster vaccinations may be administered at prescribed intervals such as every 5 to 10 years.

According to the present invention, an “effective amount” of a therapeutic composition is one which is sufficient to achieve a desired biological effect. Generally, the dosage needed to provide an effective amount of the composition will vary depending upon such factors as the subject's age, condition, sex, and extent of disease, if any, and other variables which can be adjusted by one of ordinary skill in the art. The antigenic preparations of the invention can be administered by either single or multiple dosages of an effective amount. Effective amounts of the compositions of the invention can vary from 0.01-100 μg per dose, such as from 5-50 μg per dose, or from 15-50 μg per dose. The compositions of the invention may further comprise a pharmaceutically acceptable excipient.

Definitions

When terms such as “one”, “a” or “an” are used in this disclosure they mean “at least one”, or “one or more” unless otherwise indicated. Further, the term “comprising” is intended to mean “including” and thus allows for the presence of other constituents, features, conditions, or steps than those explicitly recited.

Flavivirus as used herein refers to the genus of viruses in the family Flaviviridae. This genus includes the West Nile virus, dengue virus, tick-borne encephalitis virus, yellow fever virus, Zika virus and several other viruses which may cause encephalitis.

In this application is refered to “a sequence at least about 80% identical thereto”. This is intended to mean a variant sequence having an amino acid sequence that is substantially identical to a reference peptide, typically a native or “parent” polypeptide. The peptide variant may possess one or more amino acid substitutions, deletions, and/or insertions at certain positions within the native amino acid sequence.

The term “substantially identical” in the context of two amino acid sequences means that the sequences, when optimally aligned, such as by the programs GAP or BESTFIT using default gap weights, share at least about 80, at least about 82, at least about 84, at least about 86, at least about 88, at least about 90, at least about 92, at least about 94, at least about 96, at least about 98, or at least about 99 percent sequence identity. In one embodiment, residue positions that are not identical differ by conservative amino acid substitutions. Sequence identity is typically measured using sequence analysis software. Protein analysis software matches similar sequences using measures of similarity assigned to various substitutions, deletions and other modifications, including conservative amino acid substitutions. For instance, the publicly available GCG software contains programs such as “Gap” and “BestFit” which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences can also be compared using FASTA or ClustalW, applying default or recommended parameters. A program in GCG Version 6.1., FASTA (e.g., FASTA2 and FASTA3) provides alignments and percent sequence identity of the regions of the best overlap between the query and search sequences (Pearson, Methods Enzymol. 1990; 183:63-98; Pearson, Methods Mol. Biol. 2000; 132:185-219). Another preferred algorithm when comparing a sequence to a database containing a large number of sequences from various organisms, or when deducing the sequence identity is the computer program BLAST, especially blastp, using default parameters. See, e.g., Altschul et al., J. Mol. Biol. 1990; 215:403-410; Altschul et al., Nucleic Acids Res. 1997; 25:3389-402 (1997); each herein incorporated by reference. “Corresponding” amino acid positions in two substantially identical amino acid sequences are those aligned by any of the protein analysis software mentioned herein, typically using default parameters.

An “isolated” polypeptide is a polypeptide that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of polypeptide, in the composition). Commonly, a composition of a peptide molecule will exhibit 98%-99% homogeneity for peptide molecules in the context of all present peptide species in the composition or at least with respect to substantially active peptide species in the context of proposed use. It is to be understood that by the term “isolated”, the polypeptides according to the present invention is not naturally produced flaviviral envelope E protein, such as Zika virus envelope E protein on viral or VLP surface as part of the entire virus polypeptide sequence.

In the context of the present invention, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

The term “antigen” or “antigenic polypeptides” denotes a substance of matter which is recognized by the immune system's specifically recognizing components (antibodies, T-cells).

The term “vaccine” is used for a composition comprising an immunogen and which is capable of inducing an immune response which is either capable of reducing the risk of developing a pathological condition or capable of inducing a therapeutically effective immune response which may aid in the cure of (or at least alleviate the symptoms of) a pathological condition.

The term “pharmaceutically acceptable” has its usual meaning in the art, i.e. it is used for a substance that can be accepted as part of a medicament for human use when treating the disease in question and thus the term effectively excludes the use of highly toxic substances that would worsen rather than improve the treated subject's condition.

The term “adjuvant” as used herein refers to any compound which, when delivered together or simultaneously with an antigenic polypeptide of the invention, non-specifically enhances the immune response to that antigen. Exemplary adjuvants include but are not limited to oil in water and water in oil adjuvants, aluminum-based adjuvants (e.g., AlOH, AlPO4, etc), and Montanide ISA 720.

The terms “patient” and “subject” refer to a mammal that may be treated using the methods of the present invention.

The term “multimer” or “multimers” as used herein refers to the ability of the polypeptide of the invention to form a complex of more than two polypeptide entities, such as trimers or tetramers, pentamers, hexamers (composed of three, four, five and six monomers, respectively).

The present invention relates to any Zika virus envelope E protein which multimerizes beyond dimer/trimer, while still being secreted.

As used herein the term “flaviviral envelope E protein” and in particular “Zika virus envelope E protein” refers to a part of a flavovirus essentially comprising amino acids 292-794 of SEQ ID NO:1, or a functional fragment thereof, or the corresponding amino acids of other flavivirus species including the West Nile virus, dengue virus, tick-borne encephalitis virus, yellow fever virus, and other Zika virus. It is to be understood that different isolates of flaviviral envelope E protein, such as Zika virus envelope E protein may have small amino acid variations in the sequence, such as 1, 2, 3, 4, 5, 6, 7, 8, 9, or 10 different amino acids as compared to e.g. SEQ ID NO:1.As used herein the term “flavivirus NS1 sequence”, and in particular “Zika NS1 sequence” refers to a part of a flavovirus essentially comprising amino acids 797-1148 of SEQ ID NO:1, or a functional fragment thereof, or corresponding amino acids of other flavivirus species including the West Nile virus, dengue virus, tick-borne encephalitis virus, yellow fever virus, and other Zika virus.

It is to be understood that both the isolated polypeptides and functional fragments according to the present invention are immunogenic meaning that they are capable of inducing an immune response in a subject receiving the isolated polypeptide and functional fragment.

Accordingly a specific antibody will bind both the polypeptide and immunologic fragments thereof.

Being able to secrete the E protein significantly reduces the complexity of the purification process, and is expected to lead to higher yields as well as reduced cost compared to a membrane bound E version, while still allowing the advantage of multimirization of the antigen.

The flaviviral envelope E protein multimers according to the invention is expected to be more immunogenic than a E sub-unit is only present in dimers or trimers. Flaviviral envelope E protein produced with the entire transmembrane region is expected to forms multimers as well, but can't be secreted. Accordingly in some aspects, flaviviral envelope E protein constructs of the present invention comprises only a part of the transmembrane region.

The polypeptides and constructs according to the present invention may comprise one or more of the following elements:

a) a BiP secretion signal is a signal peptide for secretion of the protein. Many eukaryotic signal peptides are functionally interchangeable however the efficiency of protein secretion is strongly affected by the specific peptide.

b) a Tandem Strep tag may be used for purification purposes and is a construction of two strep-tags with a linker in-between. The tandem design results in a tighter binding during purification and thus a final product with higher purity.

c) one or more restriction site(s) may be added for making the construct and could be changed to any compatible restriction site.

d) a Sumo-tag as an additional purification tag which may be added in case of purification issues, It is not needed for production or vaccine action.

e) a Tev protease site may be put in between Sumo and the protein for possible removal of the sumo-tag so the end product only contains the Zika Ns1.

f) a Flexible linker may be put between the purification tag and the protein to make the tag more accessible during purification.

g) a Spytag is a short peptide that can covalently bind to spycatcher. This may be put on the construct for potential future binding to a VLP containing spycatcher.

The split-protein binding system (SPBS) is characterized by the ability of two split-fragments of a protein to re-constitute into a stable covalently locked structure. An example of such a SPBS is the Spy-tag-Spy-Catcher system, which is comprised of the reactive sub-unit protein partners (Spy-Tag and Spy-Catcher) from the second immunoglobulin-like collagen adhesin domain (CnaB2) of the fibronectin-binding protein (FbaB) of Streptococcus pyogenes (PMID: 20235501). The SpyTag and SpyCatcher interact via a spontaneous isopeptide bond formation and is an example of a split-protein binding system (Zakeri, B. et al. PNAS. 2012). If one protein contain the SpyTag and another the SpyCather the two proteins will be spontaneously linked through a covalent interaction between the SpyTag and the SpyCather which ensures a high binding strength and a one-to-one interaction between the SpyTag and SpyCatcher linked proteins. The Spytag-SpyCatcher system, together with other split-protein binding systems, is described in Veggiani et al., 2014, Trends Biotechnol. October; 32(10):506-12. The SpyCatcher protein-sequence (116 aa) displays some degree of immunogenicity in vivo, which can be reduced by truncating the N-terminal part of the protein (aa 1-24) (PMID: 25434527). While non-immunogenic, the AN-SpyCatcher sequence retains full binding capacity to the 13 aa Spytag sequence (PMID: 25434527) and can therefore be used for immuno-sensitive applications such as vaccine production or chimeric immune receptors.

Any suitable split-protein binding system may be used including the K-Tag/SpyTag/SpyLigase system, the SnoopCatcher system, and the SdyCatcher system or any system described in e.g. Tan L L, Hoon S S, Wong F T (2016) Kinetic Controlled Tag-Catcher Interactions for Directed Covalent Protein Assembly. PLoS ONE 11(10): e0165074. doi:10.1371/journal.pone.0165074.

### Specific Embodiments of the Invention

As described above the present invention relates to isolated polypeptide comprising flaviviral envelope E protein, such as Zika virus envelope E protein. In some specific embodiments these isolated polypeptide comprising flaviviral envelope E protein, such as Zika virus envelope E protein has the ability to form multimers as defined herein to be complexes of more than two polypeptide entities, such as trimers or tetramers, pentamers, hexamers etc.

In some embodiments the isolated polypeptide according to the present invention is secreted when expressed in an insect cell expression system, such as in a Drosophila expression system.

In some embodiments the isolated polypeptide according to the present invention has a length of less than 700 amino acids (aa), such as less than 690 aa, 680 aa, 670 aa, 660 aa, 650 aa, 640 aa, 630 aa, 620 aa, 610 aa, 600 aa, 590 aa, 580 aa, 570 aa, 560 aa, 550 aa, 540 aa, 530 aa, 520 aa, 510 aa, 500 aa, 490 aa, 480 aa, 470 aa, 460 aa, 450 aa, 440 aa, 430 aa, 420 aa, 410 aa, 400 aa, 390 aa, 380 aa, 370 aa, 360 aa, or 350 aa.

In some embodiments the isolated polypeptide according to the present invention has a length of more than 350 amino acids (aa), such as more than 360 aa, 370 aa, 380 aa, 390 aa, 400 aa, 410 aa, 420 aa, 430 aa, 440 aa, 450 aa, 460 aa, 470 aa, 480 aa, 490 aa, 500 aa, 510 aa, 520 aa, 530 aa, 540 aa, 550 aa, 560 aa, 570 aa, 580 aa, 590 aa, 600 aa, 610 aa, 620 aa, 630 aa, 640 aa, 650 aa, 660 aa, 670 aa, 680 aa, 690 aa, or 700 aa.

In some embodiments the isolated polypeptide according to the present invention comprises, such as at its N-terminal, at least about 80% continuous amino acids, such as at least about 85%, such as at least about 90%, such as at least about 95%, such as at least about 98% of amino acids 292-695 of SEQ ID NO:1 or a sequence at least about 80% identical thereto, such as amino acids 292-695 of SEQ ID NO:1, or amino acids 292-693 of SEQ ID NO:1.

In some embodiments the isolated polypeptide according to the present invention contains, such as at its C-terminal, at least about 10% continuous amino acids, such as at least about 20%, such as at least about 30%, such as at least about 40%, such as at least about 45% of amino acids of the N-terminal of 696-794 of SEQ ID NO:1 or a sequence at least about 80% identical thereto, such as amino acids 696-744 of SEQ ID NO:1.

In some embodiments the isolated polypeptide according to the present invention contains, such as at its C-terminal, not more than about 60% continuous amino acids, such as not more than about 50%, such as not more than about 40%, such as not more than about 30%, such as not more than about 20% of amino acids of the N-terminal of amino acids 696-794 of SEQ ID NO:1 or a sequence at least about 80% identical thereto, such as amino acids 696-744 of SEQ ID NO:1.

In some embodiments the isolated polypeptide according to the present invention consist of amino acids 292-744 of SEQ ID NO:1, or a sequence at least about 80% identical thereto, or which consist of amino acids 292-693 of SEQ ID NO:1, or a sequence at least about 80% identical thereto.

In some embodiments the isolated polypeptide according to the present invention is produced in a Drosophila S2 cell line.

In some embodiments the isolated polypeptide according to the present invention may lead to a Zika virus specific antibodies being raised, or Zika viral load reduction when used as a vaccine, or which polypeptide is suitable for protection against Zika viral infection.

In some embodiments the isolated polypeptide according to the present invention further comprises a sequence selected from the list consisting of a tag sequence for analysis and/or purification, such as a sequence of EPEA, a BiP secretion signal, a Tandem Strep tag, a Sumo-tag, a Tev protease site, a flexible linker, and a split-protein tag, such as a spy tag or Spycatcher.

In some embodiments the isolated polypeptide according to the present invention consist of SEQ ID NO:3, or a sequence with at least about 80% sequence identity thereto, or which polypeptide consist of SEQ ID NO:6, or a sequence with at least about 80% sequence identity thereto, which polypeptide is with or without the Bip signal sequence, and with or without the C-tag sequence.

Another aspect of the present invention is an isolated polypeptide comprising a flavivirus NS1 sequence, such as Zika NS1 sequence.

In some embodiments the isolated polypeptide according to the present invention is secreted when expressed in an insect cell expression system, such as in a Drosophila expression system .

In some embodiments the isolated polypeptide according to the present invention has a length of less than 700 amino acids (aa), such as less than 690 aa, 680 aa, 670 aa, 660 aa, 650 aa, 640 aa, 630 aa, 620 aa, 610 aa, 600 aa, 590 aa, 580 aa, 570 aa, 560 aa, 550 aa, 540 aa, 530 aa, 520 aa, 510 aa, 500 aa, 490 aa, 480 aa, 470 aa, 460 aa, 450 aa, 440 aa, 430 aa, 420 aa, 410 aa, 400 aa, 390 aa, 380 aa, 370 aa, 360 aa, or 350 aa.

In some embodiments the isolated polypeptide according to the present invention has a length of more than 350 amino acids (aa), such as more than 360 aa, 370 aa, 380 aa, 390 aa, 400 aa, 410 aa, 420 aa, 430 aa, 440 aa, 450 aa, 460 aa, 470 aa, 480 aa, 490 aa, 500 aa, 510 aa, 520 aa, 530 aa, 540 aa, 550 aa, 560 aa, 570 aa, 580 aa, 590 aa, 600 aa, 610 aa, 620 aa, 630 aa, 640 aa, 650 aa, 660 aa, 670 aa, 680 aa, 690 aa, or 700 aa.

In some embodiments the isolated polypeptide according to the present invention comprises, at least about 80% continuous amino acids, such as at least about 85%, such as at least about 90%, such as at least about 95%, such as at least about 98% of amino acids 1-353 of SEQ ID NO:4 or a sequence at least about 80% identical thereto, such as amino acids 1-353 of SEQ ID NO:4.

In some embodiments the isolated polypeptide according to the present invention is produced in a Drosophila S2 cell line.

In some embodiments the isolated polypeptide according to the present invention leads to a Zika viral load reduction when used as a vaccine, or which polypeptide is suitable for protection against Zika viral infection.

In some embodiments the isolated polypeptide according to the present invention further comprises a sequence selected from the list consisting of a BiP secretion signal, a Tandem Strep tag, a Sumo-tag, a Tev protease site, a flexible linker, and split-protein tag, such as a spy tag.

In some embodiments the isolated polypeptide according to the present invention consist of amino acids 1-534 of SEQ ID NO:5, or a sequence at least about 80% identical thereto.

### EXAMPLE 1

Transient Transfection

The ExpreS2 Drosophila S2 cell line was used for all work (ExpreS2ion Biotechnologies, Denmark). The day before transient transfection, cells were split by centrifugation (450×g, 3 min) and re-suspended to 8×10⁶cells/mL in EX-CELL 420 serum-free medium for insect cells (Sigma) in a 250 mL shake flask and incubated at 115 rpm at 25° C. On the day of transfection, cells were split as before to 8×10⁶cells/mL. For the transfection, 100 μL ExpreS2 Insect-TRx5 transfection reagent (ExpreS2ion Biotechnologies, Denmark) was added to 8 mL cell suspension and gently mixed, before addition of 20 μg plasmid DNA, gentle mixing and then a 5 min rest. Cells were then incubated at 25° C. and 200 rpm in a 50 mL centrifuge tube with vent cap. On day 4 the cells were harvested and the supernatant saved for analysis.

Stable Cell Line Establishment

To generate polyclonal stable cell lines, cells were split as for transient transfection on day 0. The following day, cells were resuspended to 8×106 cells/mL in EX-CELL 420 media, and 8 mL cell suspension was transferred to a 50 mL centrifuge tube with vent cap. 100 μL ExpreS2 Insect-TRx5 transfection reagent (ExpreS2ion Biotechnologies, Denmark) was added before gentle mixing and then addition of 20 μg plasmid DNA followed by gentle mixing. The transfection was then incubated at 25° C. and 200 rpm for 2-4 h before addition of 1 mL FBS. On day 2, 100 μg/mL Puromycin was added for selection. From day 4 through 14 post-transfection, the cells were counted every 3-4 days, centrifuged, and resuspended to 9×106 cells/mL in EX-CELL 420 media+10% FBS+Puromycin. After 14 days the cells were transferred directly to a 125 mL shake flask. The cells were then passaged twice by centrifugation to remove any residual Puromycin and FBS, before freezing in CryoStor CS10. In a separate 125 mL shake flask the cells were passaged twice in EX-CELL 420 without FBS before a sample of the supernatant was taken for analysis.

SDS-PAGE and Western Blots

For analysis of stable and transient transfections, samples of supernatant were prepared in 10× Bolt Sample Reducing Agent and 4× Bolt LDS Sample Buffer, and heat treated for 5 min at 95° C. Samples were run by SDS-PAGE on a Bolt 4-12% Bis-Tris Plus gel in Bolt MES SDS running buffer at 165 V for 35 min. The gels were stained with SimplyBlue SafeStain for total protein, or transferred to a nitrocellulose membrane with an iBlot Transfer Stack. Blots were stained using CaptureSelect Biotin Anti-C-tag Conjugate as primary antibody and Streptavidin-HRP as secondary antibody according to the manufacturer's protocol and detected with Novex ECL Chemiluminescent Substrate Reagent Kit. SeeBlue Plus2 Pre-Stained Marker (Life Technologies) was used.

Production

Stable cell lines expressing the Zika virus envelope E protein were expanded in shake flasks to a final volume of 2 liter. The supernatant was harvested by centrifugation and cleared by filtration through a 0.2 uM vacuum filter before storing it at minus 20 C.

Concentration

Supernatant was thawed rapidly in tepid water and cleared by filtration through a 0.2 uM vacuum filter. To access the effect of sample concentration on the Zika E1 protein the supernatant was either concentrated before chromatography or loaded directly on the column. Ultrafiltration concentrated the supernatant 15 fold using a tangential flow filtration device (Centramate, Pall) equipped with a 10 kDa MWCO membrane. Finally, the supernatant was cleared by filtration through a 0.2 uM vacuum filter.

Purification

Concentrated or un-concentrated supernatant was applied to a 3 ml Capture Select C tag (Thermo Fisher) resin and the column was washed to baseline in 20 mM TrisHCl, 150 mM NaCl pH 7.4. Bound protein was eluted using 20 mM Tris HCl, 2 M MgCl2 pH 7.4. Fractions were analysed by 10% SDS-PAGE and stained with collodial Coomassie stain (Safestain, Thermo Fisher).

Results

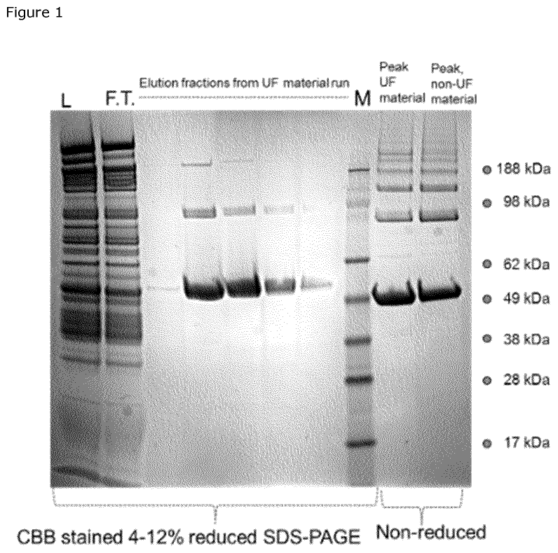

Individual fractions from the purification runs were analysed by SDS-PAGE and stained with Coomassie brilliant blue stain (CBB). In addition, non-reducing SDS-PAGE analysis of the pools of the peak containing fractions indicated multimerisation of the E protein irrespective of pre-concentration. FIG. 1 shows the result.

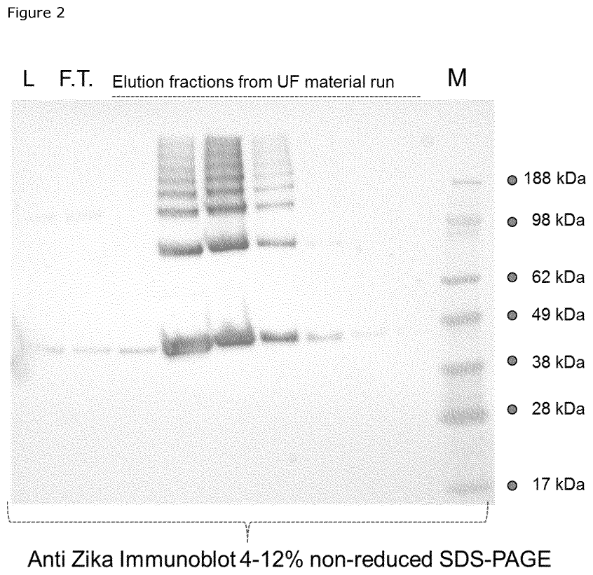

To confirm identity of the recombinant Zika virus envelope E protein a specific immunoblot using anti-Zika antibody (Aalto Bioscience catalog no. AZ 1176) was performed. Analysis of fractions from the purification of the pre-concentrated supernatant is shown in FIG. 2. A positive signal in lanes 4 -6 confirms the identity of the recombinant protein.

Conclusion

The production of a secreted, multimirizing, Zika E sub-unit antigen vaccine candidate was succesfully performed in a Drosophila S2 insect cell line. The secretion of the Zika virus envelope E protein was unexpected as almost half of the transmembrane region formed part of the E protein. Flaviriral E proteins in literature are produced either as membrane bound or secreted versions. The secreted versions are lacking the transmembrane region, while the membrane bound E proteins are produced with the transmembrane (TM) region. The Zika E sub-unit protein is with even a significant portion of the Zika virus envelope E protein TM on, secreted and multimerizing, which multimerizing may lead to enhanced immunogenicity and improved vaccine efficacy, while still allowing secretion.

The Zika E multimer was tested for its effect on Zika virus replication in a Zika mouse challenge study. The mice were injected twice with 50 ug E protein plus adjuvant, two weeks apart. The mice was infected two weeks later with Zika virus, and sacrificed four days later. Analysis of the Zika viral level in the blood, brain, and liver showed significant reductions versus the control group. This shows that the Zika E multimers function as a vaccine in a mouse challenge model, and would be expected to be an effective vaccine candidate for human use.

### EXAMPLE 2

Preparation of NS1 proteins and Ev2 constructs.

a. Upstream

The cells were scaled up in Erlenmeyer shake flasks using EX-CELLO 420 until a final volume of 2.5 l in an Optimum Growth 5 liter Flask. The cells were grown at 25° C. and shaken at 130 rpm. The supernatant was harvested by centrifugation after three days of growth and filtered through a 0.2 μm filter before storing at −20° C.

b. Downstream Sumo-Ns1-SpytSpytag (Strep Purification)

The supernatant from each construct was concentrated approximately 6-fold by tangential flow filtration (TFF, 10 kDa MWCO Pall) and then buffer exchanged into binding buffer by diafiltration. Capture, using 5 ml Streptactin-XT (IBA-GMBH) equilibrated in binding buffer, followed. The proteins were eluted from the streptactin column in Buffer BXT, Biotin Elution Buffer by Ibaiba. The pooled fractions from capturing were concentrated by spin concentrator (15 ml centriprep 30 kDa MWCO, Millipore) before loaded on a 120 ml SD200 SEC column equilibrated in 1× PBS. The final purified protein is in 1× PBS.

c. Downstream EV2 (C-tag purification) The supernatant from each construct was concentrated approximately 6-fold by tangential flow filtration (TFF, 10 kDa MWCO Pall), cleared by vacuum filtration (PES, 0.2 uM, Sigma) and then loaded onto Capture-Select C tag resin (Thermo Fischer). The column was washed to baseline OD280 absorption with 20 mM TrisHCl 150 mM NaCl pH 7.2. Elution using 20 mM TrisHCl 2 M MgCL2 pH 7.2 followed. 5 ml Streptactin-XT (IBA-GMBH) equilibrated in binding buffer, followed. The pooled fractions from capturing were concentrated by spin concentrator (15 ml centriprep 30 kDa MWCO, Millipore) before being loaded on a 120 ml SD200 SEC column equilibrated in 1× PBS. The final purified protein is in 1× PBS.

### EXAMPLE 3

The E protein from Zika virus was expressed in Drosophila S2 cells as a fusion protein with Spycatcher (Samuel C Reddington and Mark Howarth, Secrets of a covalent interaction for biomaterials and biotechnology: SpyTag and SpyCatcher; Current Opinion in Chemical Biology Volume 29, December 2015, Pages 94-99). The Spy-Tagged AP205 VLP (expressed in and purified from e. coli) was previously develped as a platform for vaccine antigen display To couple the Zika antigen to the VLP by isopeptide bond, the purified fusion protein is simply mixed with the purified VLP. Isopeptide conjugation between spycatcher on the antigen and Spy tag on the VLP N and/or C-terminus now takes place as described (Christoph M. Janitzek et al., “Bacterial Superglue Generates a Full-Length Circumsporozoite Protein Virus-like Particle Vaccine Capable of Inducing High and Durable Antibody Responses,” Malaria Journal 15, no. 1 (2016), doi:10.1186/s12936-016-1574-1).

The complex and any unreacted VLP is then subjected to density sedimentation ultra-centrifugation to purify non-aggregated particular VLP-Antigen conjugate from reaction input.

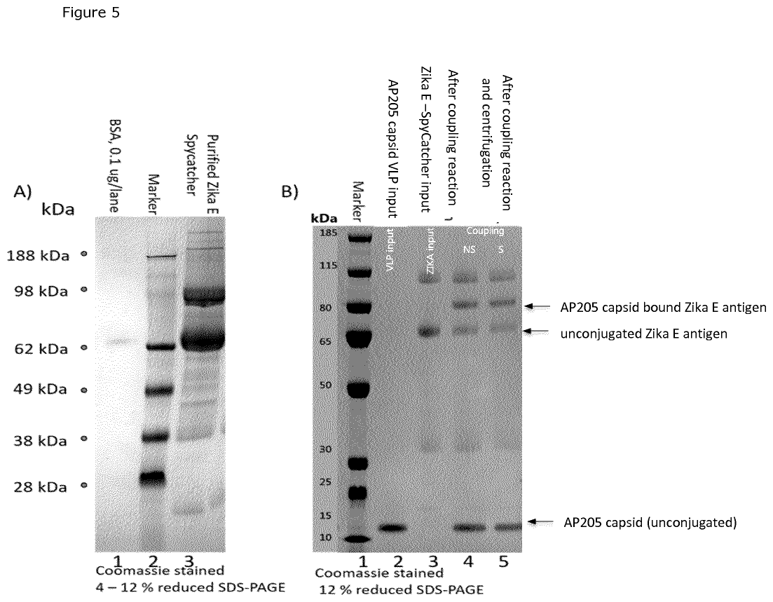

FIG. 5 clearly shows that ZIka E antigen was bound to the AP205 capsid through an isopeptide bond, leading to an extra band at 80 kDa on the SDS-PAGE gel.

Sequences:

Polyprotein [Zika virus] ACCESSION ALX35659: (SEQ ID NO: 1)

| | |
|---|---|
| 1 | mknpkkksgg frivnmlkrg varvspfggl krlpaglllg hgpirmvlai laflrftalk |
| 61 | pslglinrwg svgkkeamel ikkfkkdlaa mlriinarke kkrrgadtsv givgllltta |
| 121 | maaevtrrgs ayymyldrnd ageaisfptt lgmnkcyiqi mdlghtcdat msyecpmlde |
| 181 | gvepddvdcw cnttstwvvy gtchhkkgea rrsrravtlp shstrklqtr sqtwlesrey |
| 241 | tkhlirvenw ifrnpgfala aaaiawllgs stsqkviylv milliapays ircigvsnrd |
| 301 | fvegmsggtw vdvvlehggc vtvmaqdkpt vdielvtttv snmaevrsyc yeasisdmas |
| 361 | dsrcptqgea yldkqsdtqy vckrtlvdrg wgngcglfgk gslvtcakfa cskkmtgksi |
| 421 | qpenleyrim lsvhgsqhsg mivndtghet denrakveit pnspraeatl ggfgslgldc |
| 481 | eprtgldfsd lyyltmnnkh wlvhkewfhd iplpwhagad tgtphwnnke alvefkdaha |
| 541 | krqtvvvlgs qegavhtala galeaemdga kgrlssghlk crlkmdklrl kgvsyslcta |
| 601 | aftftkipae tlhgtvtvev qyagtdgpck vpaqmavdmq tltpvgrlit anpviteste |
| 661 | nskmmleldp pfgdsyivig vgekkithhw hrsgstigka featvrgakr mavlgdtawd |
| 721 | fgsvggalns lgkgihqifg aafkslfggm swfsqiligt llmwlglnak ngsislmcla |
| 781 | lggvliflst avsadvgcsv dfskketrcg tgvfvyndve awrdrykyhp dsprrlaaav |
| 841 | kqawedgicg issvsrmeni mwrsvegeln aileengvql tvvvgsvknp mwrgpqrlpv |
| 901 | pvnelphgwk awgksyfvra aktnnsfvvd gdtlkecplk hrawnsflve dhgfgvfhts |
| 961 | vwlkvredys lecdpavigt avkgkeavhs dlgywiesek ndtwrlkrah liemktcewp |
| 1021 | kshtlwtdgi eesdliipks lagplshhnt regyrtqmkg pwhseeleir feecpgtkvh |
| 1081 | veetcgtrgp slrsttasgr vieewccrec tmpplsfrak dgcwygmeir prkepesnlv |
| 1141 | rsmvtagstd hmdhfslgvl villmvqegl kkrmttkiii stsmavlvam ilggfsmsdl |
| 1201 | aklailmgat faemntggdv ahlaliaafk vrpallvsfi franwtpres mllalascll |
| 1261 | qtaisalegd lmvlingfal awlairamvv prtdnitlai laaltplarg tllvawragl |
| 1321 | atcggfmlls lkgkgsvkkn lpfvmalglt avrlvdpinv vglllltrsg krswppsevl |
| 1381 | tavglicala ggfakadiem agpmaavgll ivsyvvsgks vdmyieragd itwekdaevt |
| 1441 | gnsprldval desgdfslve ddgppmreii lkvvlmticg mnpiaipfaa gawyvyvktg |
| 1501 | krsgalwdvp apkevkkget tdgvyrvmtr rllgstqvgv gvmqegvfht mwhvtkgsal |
| 1561 | rsgegrldpy wgdvkqdlvs ycgpwkldaa wdghsevqll avppgerarn iqtlpgifkt |
| 1621 | kdgdigaval dypagtsgsp ildkcgrvig lygngvvikn gsyvsaitqg rreeetpvec |
| 1681 | fepsmlkkkg ltvldlhpga gktrrvlpei vreaiktrlr tvilaptrvv aaemeealrg |
| 1741 | lpvrymttav nvthsgteiv dlmchatfts rllqpirvpn ynlyimdeah ftdpssiaar |
| 1801 | gyistrvemg eaaaifmtat ppgtrdafpd snspimdtev evperawssg fdwvtdhsgk |
| 1861 | tvwfvpsvrn gneiaacltk agkrviqlsr ktfetefqkt khqewdfvvt tdisemganf |
| 1921 | kadrvidsrr clkpvildge rvilagpmpv thasaaqrrg rigrnpnkpg deylygggca |
| 1981 | etdedhahwl earmlldniy lqdgliasly rpeadkvaai egefklrteq rktfvelmkr |
| 2041 | gdlpvwlayq vasagitytd rrwcfdgttn ntimedsvpa evwtrhgekr vlkprwmdar |
| 2101 | vcsdhaalks fkefaagkrg aafgvmealg tlpghmterf qeaidnlavl mraetgsrpy |
| 2161 | kaaaaqlpet letimllgll gtvslgiffv lmrnkgigkm gfgmvtlgas awlmwlseie |
| 2221 | pariacvliv vflllvvlip epekqrspqd nqmaiiimva vgllglitan elgwlertks |
| 2281 | dlshlmgrre egatigfsmd idlrpasawa iyaalttfit pavqhavtts ynnyslmama |
| 2341 | tqagvlfgmg kgmpfyawdf gvpllmigcy sqltpltliv aiillvahym ylipglqaaa |
| 2401 | araaqkrtaa gimknpvvdg ivvtdidtmt idpqvekkmg qvlliavavs sailsrtawg |
| 2461 | wgeagalita atstlwegsp nkywnsstat slcnifrgsy lagasliytv trnaglvkrr |
| 2521 | gggtgetlge kwkarlnqms alefysykks gitevcreea rralkdgvat gghavsrgsa |
| 2581 | klrwlvergy lqpygkvidl gcgrggwsyy aatirkvqev kgytkggpgh eepvlvqsyg |
| 2641 | wnivrlksgv dvfhmaaepc dtllcdiges ssspeveear tlrvlsmvgd wlekrpgafc |
| 2701 | ikvlcpytst mmetlerlqr ryggglvrvp lsrnsthemy wvsgaksnti ksysttsqll |
| 2761 | lgrmdgprrp vkyeedvnlg sgtravvsca eapnmkiign rierirseha etwffdenhp |
| 2821 | yrtwayhgsy eaptqgsass lingvvrlls kpwdvvtgvt giamtdttpy gqqrvfkekv |
| 2881 | dtrvpdpqeg trqvmsmvss wlwkelgkhk rprvctkeef inkvrsnaal gaifeeekew |
| 2941 | ktaveavndp rfwalvdker ehhlrgecqs cvynmmgkre kkqgefgkak gsraiwymwl |
| 3001 | garflefeal gflnedhwmg rensgggveg lglqrlgyvl eemsripggr myaddtagwd |
| 3061 | trisrfdlen ealitnqmek ghralalaii kytyqnkvvk vlrpaekgkt vmdiisrqdq |
| 3121 | rgsgqvvtya lntftnlvvq lirnmeaeev lemqdlwllr rsekvtnwlq sngwdrlkrm |
| 3181 | avsgddcvvk piddrfahal rflndmgkvr kdtqewkpst gwdnweevpf cshhfnklhl |
| 3241 | kdgrsivvpc rhqdeligra rvspgagwsi retaclaksy aqmwqllyfh rrdlrlmana |
| 3301 | icssvpvdwv ptgrttwsih gkgewmtted mlvvwnrvwi eendhmedkt pvtkwtdipy |
| 3361 | lgkredlwcg slighrprtt waenikntvn mvrriigdee kymdylstqv rylgeegstp |
| 3421 | gvl |

Amino acids 292..592: region_name=“Flavi_glycoprot”; note=“Flavivirus glycoprotein, central and dimerization domains”

Amino acids 601-693: region_name=“Flavi_E_C”; note=“Immunoglobulin-like domain III (C-terminal domain) of Flavivirus envelope glycoprotein E”

Amino acids 698-794: region_name=“flavi_E_stem”; note=“flavivirus envelope glycoprotein E, stem/anchor”

Amino acids 797-1148: region_name=“Flavi_NS1”; “Flavivirus non-structural Protein NS1.

DNA Sequence of ExpreS2ion Ev1 (SEQ ID NO: 2):
atgaagctgtgcatcctgctggccgtggtggccttcgtgggactgagtct
gggacgctgcatcggcgtgtccaacCGCGATTTCGTGGAGGGCATGAGCG
GCGGAACCTGGGTGGACGTGGTGCTGGAGCATGGCGGATGCGTGACCGTG
ATGGCCCAGGATAAGCCCACCGTGGATATCGAGCTCGTGACCACCACCGT
GTCGAACATGGCCGAAGTGCGCAGCTACTGCTACGAGGCCAGCATCAGCG
ATATGGCCAGCGATAGCCGCTGCCCAACCCAGGGCGAGGCCTACCTGGAT
AAGCAGAGCGATACCCAGTACGTGTGCAAGCGCACCCTGGTGGATCGCGG
CTGGGGAAATGGATGCGGCCTGTTCGGAAAGGGCAGCCTCGTGACCTGCG
CCAAGTTCGCCTGCAGCAAGAAGATGACCGGCAAGAGCATCCAGCCCGAG
AACCTGGAGTACCGCATCATGCTGAGCGTGCACGGCTCCCAGCACAGCGG
CATGATCGTGAACGATACCGGCCACGAGACCGATGAGAACCGCGCCAAGG
TGGAGATCACCCCCAATAGTCCACGCGCCGAGGCCACGCTGGGAGGATTT
GGAAGTCTGGGCCTGGATTGCGAGCCACGCACCGGACTGGATTTCAGCGA
TCTGTACTACCTGACCATGAACAACAAGCACTGGCTGGTGCACAAGGAGT
GGTTCCACGATATCCCCCTGCCCTGGCACGCCGGAGCCGATACCGGAACC
CCACACTGGAACAACAAGGAGGCCCTGGTGGAGTTCAAGGATGCCCACGC
CAAGCGCCAGACCGTGGTGGTGCTGGGAAGCCAGGAGGGCGCCGTGCATA
CCGCCCTGGCCGGAGCCCTGGAGGCCGAGATGGATGGCGCCAAGGGACGC
CTGAGTAGCGGCCATCTGAAGTGCCGCCTGAAGATGGATAAGCTGCGCCT
GAAGGGCGTGTCCTACAGCCTGTGCACCGCCGCCTTCACCTTCACCAAGA
TCCCAGCCGAGACCCTGCACGGCACCGTGACGGTGGAGGTGCAGTATGCC
GGAACCGATGGCCCCTGCAAGGTGCCAGCCCAGATGGCCGTGGACATGCA
GACCCTGACCCCAGTGGGCCGCCTGATCACCGCCAATCCAGTGATCACCG
AGAGCACCGAGAACAGCAAGATGATGCTGGAGCTGGATCCCCCCTTCGGC
GATTCCTACATCGTGATCGGCGTGGGCGAGAAGAAGATCACCCACCACTG
GCACCGCAGCGGCAGCACCATTGGAAAGGCCTTCGAGGCCACCGTGCGCG
GAGCCAAGCGCATGGCCGTGCTGGGCGATACCGCCTGGGATTTCGGAAGC
GTGGGAGGCGCCCTGAACAGCCTGGGCAAGGGCATTCACCAGAtcttcgg
agccgcctttaagGAGCCCGAGGCCTAA
Amino Acid Sequence of ExpreS2ion Ev1, 475 amino
acids (SEQ ID NO: 3):
MKLCILLAVVAFVGLSLGRCIGVSNRDFVEGMSGGTWVDVVLEHGGCVTV
MAQDKPTVDIELVTTTVSNMAEVRSYCYEASISDMASDSRCPTQGEAYLD
KQSDTQYVCKRTLVDRGWGNGCGLFGKGSLVTCAKFACSKKMTGKSIQPE
NLEYRIMLSVHGSQHSGMIVNDTGHETDENRAKVEITPNSPRAEATLGGF
GSLGLDCEPRTGLDFSDLYYLTMNNKHWLVHKEWFHDIPLPWHAGADTGT
PHWNNKEALVEFKDAHAKRQTVVVLGSQEGAVHTALAGALEAEMDGAKGR
LSSGHLKCRLKMDKLRLKGVSYSLCTAAFTFTKIPAETLHGTVTVEVQYA
GTDGPCKVPAQMAVDMQTLTPVGRLITANPVITESTENSKMMLELDPPFG
DSYIVIGVGEKKITHHWHRSGSTIGKAFEATVRGAKRMAVLGDTAWDFGS
VGGALNSLGKGIHQIFGAAFKEPEA*

Underlined sequences are Bip signal

Bold sequences are C-tag sequence

Italic TAA/* is Stop signal

| Size | 51.00 kDa |
|---|---|
| PI | 6.20 |
| Extinction Coefficient | 1.2060 |

Zika NS1 sequence, 353 amino acids
(SEQ ID NO: 4)
VGCSVDFSKKETRCGTGVFVYNDVEAWRDRYKYHPDSPRRLAAAVKQAWE
DGICGISSVSRMENIMWRSVEGELNAILEENGVQLTVVVGSVKNPMWRGP
QRLPVPVNELPHGWKAWGKSYFVRAAKTNNSFVVDGDTLKECPLKHRAWN
SFLVEDHGFGVFHTSVWLKVREDYSLECDPAVIGTAVKGKEAVHSDLGYW
IESEKNDTWRLKRAHLIEMKTCEWPKSHTLWTDGIEESDLIIPKSLAGPL
SHHNTREGYRTQMKGPWHSEELEIRFEECPGTKVHVEETCGTRGPSLRST
TASGRVIEEWCCRECTMPPLSFRAKDGCWYGMEIRPRKEPESNLVRSMVT
AGS

Expressed in the context of the Zika NS1 construct (534aa) (SEQ ID NO:5):

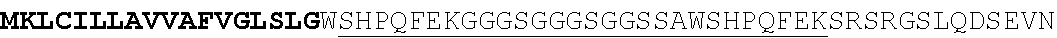

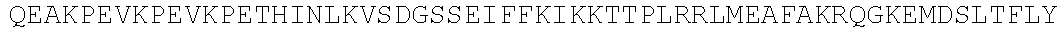

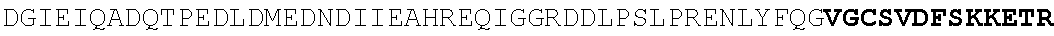

CGTGVFVYNDVEAWRDRYKYHPDSPRRLAAAVKQAWEDGICGISSVSRMENIMWRSVEGE
LNAILEENGVQLTVVVGSVKNPMWRGPQRLPVPVNELPHGWKAWGKSYFVRAAKTNNSFV
VDGDTLKECPLKHRAWNSFLVEDHGFGVFHTSVWLKVREDYSLECDPAVIGTAVKGKEAV
HSDLGYWIESEKNDTWRLKRAHLIEMKTCEWPKSHTLWTDGIEESDLIIPKSLAGPLSHH
NTREGYRTQMKGPWHSEELEIRFEECPGTKVHVEETCGTRGPSLRSTTASGRVIEEWCCR

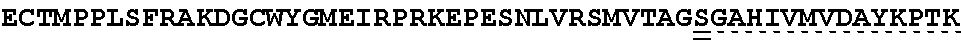

BiP secretion signal (bold first 18 aa)

Tandem Strep tag (Underlined)

Retriction site (SR and PR)

Sumo-tag

Tev protease site

Zika NS1 (Bold from VGC . . . VTAGS)

Flexible linker (G with double underline)

Spy tag (Doted line under)

Amino Acid Sequence of ExpreS2ion Ev2, 402 amino acids (SEQ ID NO:6):

MKLCILLAVVAFVGLSLGRCIGVSNRDFVEGMSGGTWVDVVLEHGGCVTV
MAQDKPTVDIELVTTTVSNMAEVRSYCYEASISDMASDSRCPTQGEAYLD
KQSDTQYVCKRTLVDRGWGNGCGLFGKGSLVTCAKFACSKKMTGKSIQPE
NLEYRIMLSVHGSQHSGMIVNDTGHETDENRAKVEITPNSPRAEATLGGF
GSLGLDCEPRTGLDFSDLYYLTMNNKHWLVHKEWFHDIPLPWHAGADTGT
PHWNNKEALVEFKDAHAKRQTVVVLGSQEGAVHTALAGALEAEMDGAKGR
LSSGHLKCRLKMDKLRLKGVSYSLCTAAFTFTKIPAETLHGTVTVEVQYA
GTDGPCKVPAQMAVDMQTLTPVGRLITANPVITESTENSKMMLELDPPFG
DSYIVIGVGEKKITHHWHRSEPEA*

Underlined sequences are Bip signal

Bold sequences are C-tag sequence

Italic TAA/* is Stop signal

1. An isolated polypeptide comprising flaviviral envelope E protein, such as Zika virus envelope E protein, which polypeptide form multimers.

2. (canceled)

3. (canceled)

4. (canceled)

5. The isolated polypeptide according to claim 1, which polypeptide comprises, such as at its N-terminal, at least about 80% continuous amino acids of amino acids 292-695 of SEQ ID NO:1 or a sequence at least about 80% identical thereto.

6. The isolated polypeptide according to claim 1, which polypeptide contains, such as at its C-terminal, at least about 10% continuous amino acids, of the N-terminal of 696-794 of SEQ ID NO:1 or a sequence at least about 80% identical thereto.

7. The isolated polypeptide according to claim 1, which polypeptide contains not more than about 60% continuous amino acids the N-terminal of amino acids 696-794 of SEQ ID NO:1 or a sequence at least about 80% identical thereto.

8. The isolated polypeptide according to claim 1, which polypeptide consist of amino acids 292-744 of SEQ ID NO:1, or a sequence at least about 80% identical thereto, or which consist of amino acids 292-693 of SEQ ID NO:1, or a sequence at least about 80% identical thereto.

9. (canceled)

10. (canceled)

11. (canceled)

12. The isolated polypeptide according to claim 1, which polypeptide consist of SEQ ID NO:3, or a sequence with at least about 80% sequence identity thereto, or which polypeptide consists of SEQ ID NO:6, or a sequence with at least about 80% sequence identity thereto, which polypeptide is with or without the Bip signal sequence, and with or without the C-tag sequence.

13. An isolated polypeptide comprising a flavivirus NS1 sequence, such as Zika NS1 sequence.

14. (canceled)

15. (canceled)

16. (canceled)

17. The isolated polypeptide according to claim 13, which polypeptide comprises, at least about 80% continuous amino acids of amino acids 1-353 of SEQ ID NO:4 or a sequence at least about 80% identical thereto.

18. (canceled)

19. (canceled)

20. (canceled)

21. The isolated polypeptide according to claim 13, which polypeptide consist of amino acids 1-534 of SEQ ID NO:5, or a sequence at least about 80% identical thereto.

22. An isolated polypeptide comprising a first part being a polypeptide comprising flaviviral envelope E protein fused to a second part being a polypeptide as defined in claim 13.

23. An isolated polypeptide according to claim 22, wherein said first part is a polypeptide as defined in claim 1.

24. (canceled)

25. A composition comprising a polypeptide as defined in claim 1 in combination with a polypeptide as defined in claim 13.

26. The isolated polypeptide according to claim 1, or a composition according to claim 25, for use in a vaccine or for use in diagnostic.

27. (canceled)

28. A conjugate comprising 1) a polypeptide selected from a) a polypeptide comprising flaviviral envelope E protein or b) a polypeptide comprising a flavivirus NS1 sequence; and 2) a Virus-like Particle.

29. The conjugate according to claim 28, wherein said polypeptide 1) is as defined in claim 1.

30. A vaccine for the protection of a subject against flavivirus infection which vaccine contains, as an active ingredient, a polypeptide according to claim 1, or a composition according to claim 25, or a conjugate according to claim 28.

31. (canceled)

32. A composition comprising a vaccine as defined in claim 30, further comprising a second flavivirus vaccine for the protection of a subject against flavivirus infection, said second flavivirus vaccine being a Virus-like Particle (VLP) or an attenuated or inactivated vaccine, or DNA vaccine.

33. A method for the protection of a subject against flavivirus infection, which method comprises administering to a subject in need of such protection an effective amount of the vaccine of claim 30.

34. An expression system for the production of an isolated polypeptide according to claim 1, which expression system comprises a first nucleotide sequence encoding said polypeptide and optionally comprising a second encoding nucleotide sequence positioned so as to produce a fusion protein wherein a secretory leader sequence is operably linked to the polypeptide, and optionally further comprising a third nucleotide sequence encoding a tag sequence for analysis and/or purification; said encoding sequences operably linked to control sequences capable of effecting expression of said encoding nucleotide sequences.

35. A eucaryotic host cell modified to contain the expression system of claim 34.
